(19) United States
(12) Patent Application Publication
(10) Pub. No.: US 2025/0082604 A1
(43) Pub. Date: Mar. 13, 2025

(54) COMPOSITIONS CONTAINING A HIGH CONTENT OF PROANTHOCYANIDINS AND METHODS OF PREPARATION AND USE THEREOF

(71) Applicant: TINTORIA PIANA, US INC., Cartersville, GA (US)

(72) Inventors: Massimo MAFFEI, Chieri (IT); Giuseppe MANNINO, Palermo (IT); Andrea PIANA, Cartersville, GA (US)

(21) Appl. No.: 18/891,337

(22) Filed: Sep. 20, 2024

Related U.S. Application Data
(63) Continuation of application No. PCT/US2022/021087, filed on Mar. 21, 2022.

(51) Int. Cl. A61K 31/353; A61K 36/15; A61K 36/45; A61K 36/48; A61K 36/87; A61P 31/04; A61P 31/14; A61P 31/16

(52) U.S. Cl. CPC A61K 31/353; A61K 36/15; A61K 36/45; A61K 36/48; A61K 36/87; A61P 31/04; A61P 31/14; A61P 31/16; A61K 2236/333; A61K 2236/51; A61K 2236/53

(57) ABSTRACT

A process of extracting proanthocyanidins (PACs) from plant materials using an ethanol or acetone solvent is described. Plant materials from one or more plant species are mixed with ethanol or acetone at a ratio of extraction solvent to plant material between 3:1 and 8:1. The mixture is then filtered and the filtrate concentrated by evaporation. The extraction process leads to an increase in the total PAC content and in the total A-type PAC content in the degree of polymerization from dimers to pentamers. Formulations containing such PAC-enriched compositions have antioxidant, antibiotic, and antiviral properties and have a negligible allergen content.

### CROSS-REFERENCE TO RELATED APPLICATION

The present application is a continuation application of International Application number PCT/US2022/021087, filed on Mar. 21, 2022. The contents of this application are incorporated herein by reference in its entirety.

### FIELD OF THE INVENTION

The invention is generally related to processes for increasing the content of proanthocyanidins (PACs), in particular A-type PACs, in plant extracts. The invention also relates to the food and medicinal use of bioactive PACs obtained from the extraction process. In particular, the invention relates to formulations based on plant extracts of different species for the preparation of tablets, capsules, softgels and any other solid preparation for the nutraceutical, pharmaceutical, and food supplement industries.

### BACKGROUND OF THE INVENTION

Plant extracts have been used for hundreds of years as a remedy for several diseases and have attracted attention due to their potential health benefits. The beneficial mechanism to prevent bacterial and viral infections has been correlated to a group of proanthocyanidins (PACs) with type A (PAC-A) bonds that exhibit bacterial anti-adhesion activity against antibiotic sensitive and resistant strains to uropathogenic Escherichia coli bacteria, including multi-resistant uropathogenic Escherichia coli.

Moreover, PAC-A bind to the external coating of some viruses (including but not limited to Herpes, Influenza, SARS, Nairovirus) by exerting an antiviral effect. In particular, high titers of low polymerization PAC-A (dimers and trimers) have a potent antiviral effect against viruses that cause herpes and flu.

Determining the optimal dose of PAC-A is central to the efficacy of plant extracts. For instance, cranberry extracts containing 72 mg of total PAC produce significant bacterial anti-adhesion activity and the percentage of bioavailable PAC-A is critical to the question of cranberry efficacy.

Plant extracts are obtained through various extraction techniques such as spray drying, a process also known as atomization. This drying process makes it possible to obtain dry powder from a liquid by rapid drying with a hot gas. This is the preferred drying technique for drying many thermally sensitive materials such as food and pharmaceuticals. Heated air is the drying medium, however, if the liquid is a flammable solvent such as ethanol or acetone or if the product is sensitive to oxygen, nitrogen is used. The use of this drying technique requires complex equipment, a large area, a significant investment, high thermal efficiency and large thermal and energy consumption. Furthermore, for the production of the fine powder, an efficient separation apparatus is required, in order to avoid product losses and consequent environmental pollution. Usually the system is inflexible and a dryer designed for fine atomization is often unable to produce a larger particle. Finally, the problem of product recovery and dust collection can significantly increase the cost. Furthermore, in many cases, the use of spray drying causes the degradation of thermolabile compounds.

Another drying process through dehydration and pulverization is freeze-drying, which is also known as lyophilization. It is a dehydration process typically used to preserve a perishable material or make the material more suitable for transportation. Freeze drying works by freezing the material and then reducing the surrounding pressure to allow the frozen water in the material to sublimate directly from the solid to the gas phase. One of the main disadvantages of freeze-drying is the high cost. In fact, the need for equipment for the freeze drying process, which requires very low temperatures, is very expensive. Freeze-drying is performed under vacuum, with absolute pressures that easily allow the ice to change directly from solid to vapor. This results in high operating costs, complex structures and machinery, high energy expenditure and significant storage problems. Also, during the freeze-drying process, some foods need to be pre-treated to prevent damage such as color fading, while other foods are damaged by the quick freeze method, and product crumbling can be an additional obstacle. Finally, even if the process allows to obtain food substances with a long shelf life, this advantage is greatly reduced if the freeze-dried substances are not stored at low humidity levels.

Another known drying process is the drying drum which is used in particular for drying liquids. For example, milk is applied as a thin film to the surface of a heated drum from which, with a blade, the remaining dried solids are scraped off. Powdered milk obtained with the drying drum tends to have a cooked taste, due to caramelization due to long exposure to heat. Compared to the techniques listed above, the drying drum is a more intense heat treatment which results in more denatured proteins. As a result, the powder is also less soluble.

Many of the known techniques also use organic matrices to facilitate the drying of the solute including in particular maltodextrins (in particular for the spray drying technique). Maltodextrins are water-soluble complex carbohydrates. They are obtained through chemical processes of partial hydrolysis of the starches of cereals (corn, oats, wheat, rice) or tubers (potatoes, yams, tapioca). A possible negative effect of the use of maltodextrin is the return hyperglycemia in consumers. Some individuals may also develop an allergic reaction to maltodextrins, particularly those with sensitivity to wheat and corn due to their similar chemical structure. Symptoms of an allergic reaction include swelling of the throat and neck, hives or skin rashes, heavy sweating and difficulty breathing.

Associated with the extraction techniques described above are complementary and preparatory techniques aimed at increasing the quantity of bioactive principles contained in plant extracts. In the case of plant extracts, the extraction of proanthocyanidins can include the following steps: a reflux of fresh or frozen fruits, shredded for one or more times at times ranging from 0.5 to 2 hours at 80-90° C. with use of an acidic (using 0.01-0.03 mol/L of organic acids such as citric acid, malic acid, quinic acid, acetic acid, benzoic acid and cinnamic acid) or non-acid solution of ethanol from 50 to 80% and subsequent recovery of the ethanol to is obtain a concentrated solution; then follows a centrifugation to obtain supernatant fractions and centrifuged slag; the supernatant is then separated through the use of ion exchange resins and macroporous resins with the use of water and ethanol (50-70%) to eliminate the excess of free sugars and to obtain concentrated fractions of anthocyanins and proanthocyanidins. The eluates are then concentrated under vacuum. After the addition of excipients (mainly maltodextrin), drying is carried out using vacuum systems or as described above. In general, extraction techniques such as the one described above allow to obtain a product with a variable total proanthocyanidin titer. The recurring values of total PAC vary from 5 to 80% on dry weight. The fraction of active proanthocyanidins of type A (from dimers to heptamers) usually does not exceed 50-80%.

New methods for enhancing the PAC and PAC-A content of plant extracts are needed.

### SUMMARY

Embodiments of the present disclosure are based on the discovery that, contrary to current understanding, the content of PAC in plant extracts can be increased. The present disclosure therefore provides an extraction process which allows to increase both the total content of PAC and the total percentage of PAC-A in plant extracts which allows to obtain a higher titer that provides the greatest bioactivity and efficacy against bacterial infections of the urogenital tract and viral infections. The extraction process utilizes ethanol or acetone which are non-harmful liquid solvents accepted by various national legislations. The process is economical, consumes a relatively low amount of energy, uses relatively low temperatures, and does not pollute the environment.

An aspect of the disclosure provides a method for preparing a PAC-enriched composition, comprising mixing a plant material containing PACs with an extraction solvent comprising 90-100 vol % ethanol or 90-100 vol % acetone to provide a mixture, filtering the mixture to provide a filtrate, and evaporating the filtrate to provide a dried PAC-enriched composition. In some embodiments, the method further comprises crushing the dried PAC-enriched composition to provide a powder. The plant material may be obtained from a variety of species such as Vaccinium macrocarpon, Vitis vinifera, Arachis hypogaea, Pinus pinaster, and Vaccinium vitis-idaea. In some embodiments, the extraction solvent further comprises an acid. In some embodiments, a volume-to-weight ratio of extraction solvent to plant material is between 3:1 and 8:1. In some embodiments, the mixing step is performed for 10-60 minutes. In some embodiments, the mixing step is performed at a temperature of 10-30° C. In some embodiments, the evaporating step is performed using vacuum evaporation, optionally followed by oven drying, or the evaporating step is performed via spray drying. The PAC-enriched composition may comprise dimeric, trimeric, tetrameric, and pentameric PACs and may be enriched for A-type PACS. In some embodiments, the method further comprises determining a PAC content in the plant material before the mixing step.

Another aspect of the disclosure provides a PAC-enriched composition prepared by a method as described herein. In some embodiments, the composition is in a solid dosage form selected from a tablet, capsule, or softgel.

Another aspect of the disclosure provides a formulation comprising PAC-enriched compositions extracted from each of Vitis vimfera, Arachis hypogaea, Pinus pinaster, Vaccinium vitis-idaea. In some embodiments, the formulation comprises 1-25 wt % Vitis vinifera PAC-enriched composition, 40-85 wt % Arachis hypogaea PAC-enriched composition, 1-35 wt % Pinus pinaster PAC-enriched composition, and 5-45 wt % Vaccinium vitis-idaea PAC-enriched composition. In some embodiments, the formulation contains 500-600 mg/g of PACs. In some embodiments, the formulation has a peanut allergen content of 1.5 μg or less.

Another aspect of the disclosure provides a method of inhibiting replication of viral particles comprising contacting the viral particles with a composition or formulation as described herein. In some aspects, the disclosure provides a method of treating a viral infection in a subject in need thereof comprising administering to the subject a therapeutically effective amount of a composition or formulation as described herein. In some embodiments, the viral infection is caused by a virus selected from the group consisting of influenza virus, respiratory syncytial virus, coronavirus, ebola virus, and human immunodeficiency virus.

Another aspect of the disclosure provides a method of inhibiting adhesion of bacteria to cells, comprising contacting the bacteria with a composition or formulation as described herein. In some aspects, the disclosure provides a method of treating a bacterial infection in a subject in need thereof, comprising administering to the subject a therapeutically effective amount of a composition or formulation as described herein. In some embodiments, the bacterial infection is a urinary tract infection caused by one or more of Escherichia coli, Coagulase negative Staphylococci, Enterococcus Spp., Helicobacter pilorii, Candida albicans, and Non-albicans Candida Spp.

The foregoing and other objects, features, and advantages of the present disclosure will become more apparent from the following detailed description, which proceeds with reference to the accompanying figures.

### BRIEF DESCRIPTION OF THE DRAWINGS

FIG. 1. BL-DMAC calibration curve after using ethanol as the extraction solvent.

FIG. 2. BL-DMAC calibration curve after using acetone as the extraction solvent.

FIG. 3. BL-DMAC calibration curve after fractionation and purification of the SP4 formulation.

FIG. 4. Dose-response curve of the SP4 formulation in human HepG2 cells.

FIG. 5. SP4 inhibits Influenza A virus replication.

FIG. 6. SP4 inhibits Influenza B virus replication.

FIG. 7. SP4 inhibits Respiratory Syncytial virus A replication.

FIG. 8. SP4 inhibits hCoV-OC43 virus replication.

### DETAILED DESCRIPTION

Embodiments of the disclosure provide extraction methods which provide an enhanced total content of PAC and an increased percentage of PAC-A in plant extracts. Compositions and formulations containing increased amounts of PAC and PAC-A are useful as antioxidant, anti-viral, and antibacterial agents.

The term “extraction” refers either to the process of extraction as a whole or to the individual step of extraction (leaching) that forms a part of the process. In the individual step of extraction, plant materials, or parts thereof, are contacted with a suitable solvent that extracts out (leaches out) one or more constituents/components thereof. Similarly, the term “extract” refers, depending on the context, either to the solution that is obtained during, and/or at the end of the extraction step, or to the solid mass that would be obtained upon removal by evaporation or otherwise, of the solvent contained in the solution. The solid mass is also sometimes referred to herein as the “solute”, which has also been used herein to refer to the one or more components of plant that are soluble in the solvent. The soluble components may be desired ones from the point of view of extraction or otherwise. The term “solvent” includes solvent mixtures unless the context requires otherwise, that is, the expression “solvent/solvent mixture” has been shortened to “solvent” in the interests of clarity and conciseness.

Many plants are known to contain PACs, and any such plants can be employed in the extraction methods described herein. PAC-containing plants are members of the Coniferiae class including plants from the order Coniferales and particularly from the family Pinaceae (including pines); members of the family Arecaceae (including palms); monocot plants including members of the orders Pandanales, Arales, Najadales, Restionales, Poales (including grains such as sorghum, barley and others), Juncalaes, Cyperales (including cypress), Typhales, Zingiverales, and Liliales (including lilies); dicot plants from the orders Laurales (including laurel, cinnamon), Fagales (including oak), Casuarinales, Dilleniales, Malviales (including cotton), Salicales, Ericales (including cranberries, blueberries, rhododendron), Ebenales, Rosales (including roses, blackberries and other related berries, apples, peaches, plums), Fabales (including legumes, wysteria), Myrtales, Proteales, Rhamanales (including grapes) and Sapindales. Exemploary plants include the dicots Ericaceae, which includes the Vaccinium spp., Rosaceae and Vitaceae, which includes the Vitis spp.; and the conifers of the Pinaceae family. The Vaccinium spp. include, but are not limited to, plants with cranberry-type berries such as V. macrocarpon (cranberry), V. vitis-idaea (mountain cranberry, cow berry, lingonberry) and V. oxycoccus (European cranberry); and plants with blueberry fruit such as V. augustifolium (low sweet blueberry), V. ashei (Rabbiteye blueberry), V. corymbosum (high bush blueberry), V. lamarckii (early sweet blueberry) and V. myrtillus (bilberry, European blueberry). The Vitis spp. include, but are not limited to, V. labrusca (Fox grape), V. rotundifolia (muscadine, scuppernong), V. vinifera (European grape) and all inter specifc hybrids with other Vitis species. Preferred embodiments include cranberry (Vaccinium macrocarpon Aiton, Ericaceae), lingonberry (Vaccinium vitis-idaea L., Ericaceae), grape seeds (Vitis vinifera L., Vitaceae), peanut skins (Arachis hypogaea L., Fabaceae), pine barks (Pinus pinaster Aiton, Pinaceae), elderberry (Sambucus nigra L., Adoxaceae), and pomegranate (Punica granatum L., Lythraceae).

The plant material can be from any part of the plant and preferably is from a part of the plant rich in PACs. For example, plant material includes leaves, fruit (both mature or ripe fruit, and immature or unripe fruit), stems, seeds, bark and roots, and can be used for preparation of the PAC extract.

When using peanut extract, the protein content of peanuts might be high. Many of these proteins are known to be allergenic, such as arachine and conarachine, which are contained in relatively high quantities. Peanut is also one of eight foods considered to be the most common cause of food allergies. Peanut allergies are among the most serious and can lead to life-threatening anaphylactic shock. Therefore, in the United States and the European Union as well as in many other countries, the indication on the label of the presence of peanuts in food is mandatory. According to regulation (EU) n. 1169/2011 the presence of peanuts or parts of it, if used as an ingredient, must be declared in food products. Similar rules exist for example in the United States, Canada, Australia and New Zealand. Peanut allergy is usually lifelong and even a trace amount of peanut allergens can cause severe anaphylactic reactions. Hence, it is important to detect peanut allergens in food products. Enzyme-linked immunosorbent assay (ELISA) is a common protein-based method that detects allergenic proteins based on the interaction of the species-specific proteins with antibodies and it is widely used by food industry and official food control agencies. The rapid, sensitive and simple DNA-based methods are an alternative assay to direct protein detection, in which some emerging methods can be run without large-scale equipment. These methods include nucleic acid amplification-based [e.g. real time polymerase chain reaction (qPCR)]. DNA-based methods are indirect detection approaches of peanut allergens based on a segment of the gene encoding allergenic protein, it is more stable than protein detection during food processing and extraction procedures. The eliciting dose (ED) for a peanut allergic reaction in 5% of the peanut allergic population, the ED₀₅, is 1.5 mg of peanut protein. In some embodiments, the formulation has a peanut allergen content of 2 μg or less, e.g. about 1.8 μg or less or about 1.5 μg or less.

Proanthocyanidins are polyphenol compounds in which flavanols are condensed or polymerized. The flavanoid polyphenolics include the catechins, anthocyanins, and proanthocyanidins. Proanthocyanidins are also known as proanthocyanins, leucoanthocyanins, anthocyanins or procyanidins; these terms can be used interchangeably. For general reviews see, Porter, “Flavans and Proanthocyanidins” in The Flavonoids: Advances in Research Since 1986 (Harborne, ed.) Chapman and Hall, London, 1993, pp. 23-55; Haslam, Chemistry of Vegetable Tannins, Academic Press, New York, New York, 1966; or Singleton and Esau, Phenolic substances in Grapes and Wine, and Their Significance (1969).

An A-type interflavanoid linkage is one which results when the flavanoid units are joined by two bonds, with one bond occurring between C4 of the “upper or first” unit and C8 of the “lower or second” unit and the other bond occurring between C2 of the upper unit and the oxygen attached to the C7 of the lower unit. This linkage leads to the formation of an additional 6-membered ring. A B-type interflavanoid bond (or linkage) is one which results when the flavanoid units are joined by a single bond. That bond occurs between either the upper unit C4 and the lower unit C8 or between the upper unit C4 and the lower unit C6.

The plant extract may contain PAC in the form of a monomer, dimer, trimer, tetramer, pentamer, hexamer, heptamer and higher oligomers. In some embodiments, the extracts are enriched for one or more of dimers, trimers, tetramers, or pentamers, preferably dimers and trimers, e.g. the final composition contains more dimers and trimers than other PAC forms. In some embodiments, the extracts are enriched for A-type PACs (PAC-A). The term “enriched” refers to a higher percentage or amount of the enriched component in the extract as compared to the percentage or amount found in the plant material prior to extraction. In some embodiments, the compositions described herein contain at least a 5% increase in PACs (i.e. total PACs, certain oligomeric forms of PAC, and/or A-type PACs), e.g. at least a 10%, 20%, 30%, 40%, 50%, 60%, 70%, 80%, 90%, 100% or higher increase as compared to the original plant material. In some embodiments, there is a 100-900% increase or more, e.g. a 200%, 300%, 400%, 500%, 600%, 700%, 800%, 900%, 1000% increase or more.

In some embodiments, the enriched composition contains at least about 200-600 mg/g of PACs, e.g. at least 250, 300, 350, 400, 450, 500, 550, 600, 650 mg/g or more. In some embodiments, the formulation contains at least about 50-200 mg/g of PAC-A per solid dosage form, e.g. at least about 75, 100, 125, 150, 175, 200, 225 mg/g or more.

Percent yield is the percent ratio of actual yield to the theoretical yield. In some embodiments, the yield of PACs (i.e. total PACs, certain oligomeric forms of PAC, and/or A-type PACs) in the extract is at least about 20%, e.g. at least about 25%, 30%, 35%, 40%, 45%, 50%, 55%, 60%, 65%, 70%, 75%, 80%, 85%, 90%, 95%, 96%, 97%, 98%, 99%, or 100%.

The extraction methods include a step of mixing (i.e. homogenizing) a plant material containing PACs with an extraction solvent comprising ethanol or acetone. The solvent may be an aqueous solvent. Either ethanol with an alcohol content of 90 to 100%, preferably 95 to 98%, more preferably 96%, or acetone 90% to 100% purity, preferably 95 to 100% purity, more preferably 100% purity is added to the plant material containing PAC with a variable volume-to-weight ratio between 3:1 and 8:1 (volume of solvent:weight of plant material), more preferably between 5:1 and 7:1, with a preferred ratio of 4.5:1. Usually, the ratio is established by the concentration of the total PACs present in the plant material. For example, if the plant material contains 250 g/kg of total PAC and you want to obtain a powder with 360 g/kg of total PAC with an average percentage of 85% of PAC-A then an extraction ratio of 4.75:1 is used.

In some embodiments, the solvent further comprises an acid, for example, in an amount of 0.1-5 vol %, preferably 1-3 vol %. Suitable acids include, but are not limited to, hydrochloric acid, formic acid, acetic acid, and phosphoric acid acid.

The term “mixing” refers to a mixing process similar to that used to prepare common solutions. The mixing is preferably carried out in special devices called mixers, built with materials compatible with food or pharmaceutical production (for example, a stainless steel Aisi 304-316 container), but can alternatively be carried out in devices capable of mixing the plant materials and the matrix continuously.

The extracts may be mixed for a period ranging from 10-100 minutes, e.g. 10-60 minutes, 30-60 minutes, or 20-30 minutes. The mixing preferably takes place at room temperature preferably between 1° and 30° C., for example 20-25° C. or 20-22° C. until the homogeneity of the product is achieved.

A mixing blade can rotate clockwise or counterclockwise. The speed of rotation of the blade is preferably less than 100 revolutions per minute, for example between 5 and 50 revolutions per minute, even more preferably less than 30 revolutions per minute, for example 20 revolutions per minute. The preferred rotation speed of the blade is preferably between 50 and 150 revolutions per minute, more preferably around 90 revolutions per minute but can reach, on the basis of the treated matrices, a maximum of 200 revolutions per minute.

The extraction methods may also include one or more optional pre-treatment operations such as washing, cleaning, soaking, drying, cutting, chopping, blanching, and others, if necessary.

In some embodiments, before the mixing step, a chemical characterization step is performed. Four methods have been used to evaluate the total PAC content in plant extracts. Two methods are based on PAC depolymerization (for example, the butanol and hydrochloric acid method known as Bates-Smith and the Pharmacopoea Europaea method). Two other methods are colorimetric methods (a UV-Vis spectrophotometric method based on Prussian blue reagents or Folin-Ciocalteu reagents and the BL-DMAC method). The BL-DMAC (an aldehyde condensation of 4-dimethylaminocinnamaldehyde) colorimetric method is the most accurate compared to the other methods and has been used successfully to quantify PACs in plant extracts. The BL-DMAC method is less likely to be subject to interference from other typical plant components of plant extracts, such as anthocyanins, because the reaction is read at a wavelength of 640 nm, where these compounds do not absorb. However, the BL-DMAC method, although specific for the quantification of the total PAC content, is unable to distinguish between A type and B type PAC; therefore, analytical methods such as HPLC coupled to mass spectrometry, fluorescence detectors or analytical systems such as MALDI-qTOF-MS are required for the precise identification and quantification of A type PAC.

Subsequently, the mixture is subjected to filtration which may involve the use of filtering systems such as, but not limited to, a Buchner filter to carry out vacuum filtration; absorbent paper or silica gel filter; a filter press, that is a system of volumetric reduction of liquid substances that have suspended solids inside and composed of a series of plates or plates alternating with filters adhering to each other to form chambers, in which the solute cake is formed and dehydrated; or separation by means of a solid ejection centrifuge, with or without a solid discharge, which is used to remove suspended solids from a liquid, but can also act as a purifier and concentrator to remove suspended solids, while separating two mixed and immiscible liquid phases of different densities.

Regardless of the method used for filtration, the phase enriched with total PAC and PAC-A is the liquid phase obtained from filtration, while the solid residue contains smaller quantities of total PAC and PAC-A.

The liquid phase may then be concentrated through evaporation which removes most or all of the solvent. The evaporation may be performed using a vacuum evaporation system in order to concentrate the solute (containing the total PAC and PAC-A) and recover the solvent (ethanol or acetone). This operation allows to recover the solvent and reduce the environmental impact of the evaporative process. Preferably the system operates at temperatures between 30-90° C., e.g. 35-50° C., more preferably at about 40° C. and with a pressure of about 10-30 millibars, e.g. about 20 millibars.

Alternatively, the filtrate can be dried with vacuum evaporation until a dense liquid is reached which is then placed on trays that are subjected to preliminary ventilation under an extractor hood to eliminate most of the solvent used and subsequently subjected to a drying phase in a vacuum oven with temperatures between 40-90° C., preferably between 50-80° C., more preferably at about 70° C. and a pressure level between 0 and 200 mb, preferably between 0 and 80 mb, more preferably 30 mb. The drying time can vary from 2 to 12 hours, preferably from 4 to 10 hours, more preferably for about 6 hours.

Alternatively, obtaining the powder from the extract can be obtained by means of an atomizer (spray-dryer), used for the nebulization and drying of liquid suspensions. The material to be dried enters the form of a high pressure liquid inside a distribution ring equipped with a certain number of outlets (atomization lances). As a result of the pressure, the liquid escapes from the nozzles in the form of small droplets (atomization) which increases the heat exchange due to the increase in the specific surface. The droplets meet the hot air that is introduced, the liquid evaporates very quickly and moves away in the form of vapor, while the solid contained within each drop forms agglomerates, generally hollow inside due to fast drying.

The dried material may then be crushed with a mill in order to form a powder. The degree of crushing may vary according to the final use of the powder. A “powder” is defined as a material divided into particles with a diameter between approximately 1 and 1000 micrometers (or microns). Pulverization is the method used for crushing dehydrated substances into particles called dust. Typically 0.5-1 mm sieves are used. The pulverization time depends on the crushing speed of the mill.

Subsequently, the ground product can be collected in containers with bags and then stored in a cool, dry place protected from light.

The extraction process can then be validated by analyzing the PAC content. Preferably, the pulverized product sample is extracted and analyzed by liquid chromatography in combination with mass spectrometry. An analysis on the total content of PAC can also be carried out using the BL-DMAC method. The experimental values can confirm the increase in the concentration of total PAC and the increase in the percentage of PAC-A, with particular reference to dimers and trimers. Furthermore, the analysis can indicate the absence of alterations in the chemical composition.

Further embodiments provide a formulation comprising PAC-enriched compositions extracted from two or more plant species. A preferred embodiment provides a formulation comprising PAC enriched compositions extracted from two or more of Vitis vinifera, Arachis hypogaea, Pinus pinaster, Vaccinium vitis-idaea. In some embodiments, the formulation comprises 1-25 wt % Vitis vinifera PAC-enriched composition, 40-85 wt % Arachis hypogaea PAC-enriched composition, 1-35 wt % Pinus pinaster PAC-enriched composition, and 5-45 wt % Vaccinium vitis-idaea PAC-enriched composition. In some embodiments, the formulation contains at least about 200-600 mg/g of PACs, e.g. at least 250, 300, 350, 400, 450, 500, 550, 600, 650 mg/g or more. In some embodiments, the formulation contains at least about 50-200 mg/g of PAC-A per solid dosage form, e.g. at least about 75, 100, 125, 150, 175, 200, 225 mg/g or more.

It is clear from the previous description that the process according to the disclosure allows to obtain numerous advantages and to increase the quantity and quality of the bioactive compounds extracted, with particular reference to PAC-A dimers and trimers. In particular, the process makes it possible to use plant extracts with solvents compatible with current regulations, in particular for example the EEC directive 2009-32. Furthermore, the process described allows the use of fluids considered acceptable for the preparation of foods such as ethanol, or acetone which is particularly suitable when ethanol is not allowed by some regulations (e.g., Halal). Furthermore, the process described allows to use all the extracts deriving from extraction processes with supercritical fluids, such as for example CO₂.

In particular, the process is economical and does not require particularly expensive equipment, consumes a relatively low amount of energy, does not pollute the environment and uses relatively low temperatures (e.g. from 20 to 25° C.) for the mixing phase. In addition, the process operates at relatively low temperatures also for the evaporation of residual solvents (e.g. from 40 to 60° C., or lower if a vacuum system is used) without inducing alterations or degradations of the substances present in the extract. Furthermore, the process uses extracts of natural substances included in the lists of substances admitted by the FDA and other regulatory bodies and is not allergenic. No solvents are used other than those already present in the liquids to be dried (in the case of re-hydration or resuspension of dry extracts, only ethanol or acetone are used).

Another advantage of the present invention is the possibility to combine different plant extracts with an increased PAC-A content in a calibrated formulation that allows the preparation of tablets, capsules, softgels and any other solid preparation to be used as food supplements of proved efficacy. “Food supplements” are defined as food products intended to supplement the common diet and which constitute a concentrated source of nutrients, such as vitamins and minerals, or of other substances having a nutritional or physiological effect, in particular, but not exclusively, amino acids, essential fatty acids, fibers and extracts of vegetable origin, both single and multi-compound, in pre-dosed forms. Food supplements are also defined as those products aimed at promoting the intake of certain nutrients not present in the foods of an incomplete diet recommended in cases where the body has a shortage of certain foods.

Furthermore, the present disclosure provides pharmaceutical compositions comprising a PAC-enriched extract together with a pharmaceutically or nutraceutically acceptable carrier. “Pharmaceutical composition” or “Neutraceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound as described herein and a pharmaceutically acceptable carrier.

The active agent may be combined with pharmaceutically acceptable excipients. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The compositions and formulations of the present disclosure may also contain other components such as, but not limited to, additives, adjuvants, buffers, tonicity agents, and preservatives. An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, saccharin, trehalose, mannose, D-galactose, and lactose and flavorings such as orange oil or other essential oils.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

A pharmaceutical composition or formulation may additionally comprise one or more other compounds as active ingredients like one or more additional compounds of the present disclosure, one or more antioxidant agents, antiviral agents, or antibacterial agents.

The compositions and formulations include compositions suitable for oral, rectal, topical, ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

The composition and formulations can be provided, e.g., in a solid dosage form such as tablets, capsules, softgels or liquid form, oral rinse, douche, topical formulation, toothpaste or as an additive for a beverage or other food item.

The PAC compositions or formulations can be used directly as food additives or mixed with a consumable carrier to be used as a food additive or food composition. The food compositions thus contain one of the PAC compositions in admixture with livestock feed, domestic animal feed or with a consumable food product. Those food compositions which contain livestock feed are for cattle, pigs, turkeys, chickens and the like. Those food compositions which contain domestic animal feed are for dogs, cats, horses and the like. Those food compositions which contain a consumable food product are for mammals, preferably for humans and primates. The food compositions, especially beverages, can be used as a support against bacterial (e.g. urogenital) or viral infections. Alternatively, the food compositions can be general consumables, for example, ground meat or other meat product, beverages, especially juice beverages, whether or not pasteurized, grain products, fruit products and the like.

In some embodiments, the extract compositions/formulations are substantially free of anthocyanins, flavonols, hydrolyzable tannins, alkaloids, lipids, carbohydrates, simple sugars, protein and amino acids, alcohols and organic acids. The presence or absence of these compounds can be determined by standard chemical testing, measures of purity or other conventional means known in the art. The PAC compositions and formulations may be substantially pure. The term “substantially pure” refers to PACs having a purity, measured as % area HPLC, of about 95% or more, e.g. 96%, 97%, 98%, 99% or more.

The compositions and formulations described herein are useful as antibacterial agents. Without being bound by theory, it is believed that PACs are similar in structure to the bacterial-binding receptors found on the surface of bladder or kidney cells. These compounds may act, not by killing the bacteria directly, but rather by binding bacterial fimbriae and thereby preventing adherence of the bacteria to bladder or kidney cell surface receptors. Alternatively, PACs may inhibit biosynthesis of the bacterial fimbriae, without which adherence cannot occur. No matter the mechanism, if the bacteria cannot bind to the cells, they cannot multiply, and these are two steps apparently necessary to cause a urinary tract infection. The bacteria are thus carried harmlessly out of the body in the urine stream. This anti-adherence property is advantageous since it eliminates the selective pressure to develop antibiotic resistance that can occur during multiplication of bacteria in the presence of antibiotics and limit the so-called recurrence, which occurs when two or more episodes of cystitis develop in one year.

Embodiments include in vitro methods of inhibiting adhesion of bacteria to cells, comprising contacting the bacteria with a composition or formulation as described herein. Further embodiments provide in vivo methods of treating a bacterial infection, e.g. a urinary tract infection, in a subject in need thereof, comprising administering to the subject a therapeutically effective amount of a composition or formulation as described herein. In some embodiments, the bacteria is one or more of Escherichia coli, Coagulase negative Staphylococci, Enterococcus Spp., Helicobacter pilorii, Candida albicans, and Non-albicans Candida Spp.

Embodiments may include a method of inhibiting adherence of bacteria to a surface which comprises contacting said bacteria with a composition or formulation as described herein, prior to or concurrently with contacting said bacteria with said surface. The surface can be any substance or material, synthetic or biological, where it is desired to prevent bacterial contamination, accumulation or infection. The surface can also be or constitute a biofilm. In a preferred embodiment, the surface is a cellular surface such as an uroepithelial cell surface, cells exposed in a wound or on the skin or another surface such as teeth or a prosthetic device or implant or a biofilm on any of these objects.

Treatment in accordance with the disclosure renders bacteria non-pathogenic and unable to colonize the urinary tract. Thus, one measure of efficacy includes monitoring the reduction or elimination of urinary bacterial counts associated with such infections during or after the course of treatment. Prevention in accordance with the invention does not mean complete prevention of infection in any particular individual but rather means a statistical reduction in the incidence of urogenital infections in a population sample.

The compositions and formulations described herein are useful as antiviral agents. Embodiments include in vitro methods of inhibiting replication of viral particles comprising contacting the viral particles with a composition or formulation as described herein. Further embodiments provide in vivo methods of treating a viral infection in a subject in need thereof comprising administering to the subject a therapeutically effective amount of a composition or formulation as described herein. In some embodiments, the viral infection is caused by a virus selected from the group consisting of influenza virus (e.g. influenza type A and B), respiratory syncytial virus, coronavirus, ebola virus, human immunodeficiency virus, herpes virus (e.g. herpes simplex 1 and 2 and herpes zoster), nairovirus, reovirus, and enterovirus.

The PAC compositions and formulations can also be used for reducing or treating infection after surgery, treating topical wounds or acne, or preventing or eliminating oral infection (e.g. but not limited to canker sores, gingivitis, caries) by administering a composition or formulation as described herein to a site of infection or potential infection in a patient. The composition is administered to the patient in accordance with the treatment being rendered. For example, it can be applied to a surgical incision or other opening as a liquid, topical cream or by any other suitable delivery means. For topical wounds, the composition can be a tropical cream, salve or spray. Oral infection can be treated by brushing with a toothpaste or by using a oral rinse or mouth wash formulated with PACs in accordance with the disclosure.

A patient or subject to be treated by any of the compositions or methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the active agent is administered to the subject in a therapeutically effective amount. By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from about 1 mg to about 500 mg per day, preferably from about 10 to about 250 mg per day and more preferably from about 25 to 100 mg per day.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example 1. Increasing the PAC Content of a Plant Extract Containing PACs by Using Ethanol as a Solvent

### Materials and Methods

A method for increasing the concentration of total PAC and the percentage of PAC-A (with particular reference to dimers and trimers) in commercial samples with a PAC content is described below.

Increased total PAC content starting from American cranberry, grape seed, peanut skin, pine bark and lingonberry extracts.

Before proceeding, the chemical characterization of the extracts is performed through assays on the total content of PAC (BL-DMAC assay) and the chemical characterization of PAC-A is done by liquid chromatography combined with mass spectrometry.

The quantification of the total PACs is performed by the BL-DMAC spectrophotometric assay. The BL-DMAC spectrophotometric assay is strongly influenced by interferents present in the glassware and in the material used during the extraction and assay. It is therefore necessary to use disposable plastic material. Sampling tubes must be washed thoroughly with detergents (polyphosphates), immediately rinsed and immediately washed with distilled water with 10% HCl. After rinsing with distilled water, they can be left to dry in the oven at 80° C.

For the preparation of solvents and standards the following protocol is used:
1. PAC extraction solvent (for characterization of PAC content before extraction with ethanol solvent): Transfer 75 mL of acetone into a glass cylinder and add 24.5 ml of distilled water and 0.5 ml of acetic acid. Transfer the extraction solvent into 2 50 ml plastic tubes. This solution is stable for 1 year at 18-25° C.
2. Acidified Ethanol: Concentrated hydrochloric acid (36%) (12.5 mL) is added to 12.5 mL of distilled water and 75 mL of ethanol (96.6%) in a glass cylinder. This solution is stable for 1 year at 18-25° C.
3. DMAC Reagent: Prepare a 0.1% w/v solution of DMAC (4-DiMethylAminoCinnamaldehyde) in acidified ethanol. Weigh 0.05 g of DMAC and add 50 mL of acidified ethanol. This reagent must be prepared fresh daily. Keep the reagent in the dark on ice. 4. PAC stock solutions: Prepare a stock solution of PAC-A2 and PAC-B2 at the final concentration of 100 μg ml⁻¹. Dissolve the powder (5 mg) in 50 ml of ethanol (96.6%) (Warning: DO NOT use the acidified ethanol solution). Shake by vortexing and check for the absence of sediments. Divide the solution into aliquots of 500 μl each in 1.5 ml Eppendorf tubes. Store the aliquots at −20° C. for 6 months or at −80° C. for longer times.

### Pre-Characterization of PAC Content

Using an analytical balance, 100 milligrams of plant extract for each species is weighed. The extract is then placed in a 15 ml glass tube with a screw cap. Then proceed to add 10 ml of extraction solvent, shake by vortex, extract for 30 min in an ultrasonic bath in the dark and at room temperature, shake the sample for 1 hour on a tilting system, centrifuge for 5 min at 5000 g. Finally, the samples must be kept in the dark on ice until the moment of analysis.

The spectrophotometric assay is therefore performed in the presence of a high concentration of hydrochloric acid capable of catalyzing the formation of carbocation. Acidified ethanol has a high viscosity so it is necessary to slowly empty the plastic tip to allow the solvent film to drain completely from the tip. The samples are then adequately diluted in Eppendorf tubes. For the dilution of the samples, use the extraction solvent. The sample is then diluted 1:100 by proceeding as follows. Pipette the sample aliquot into the dilution solvent; then close the Eppendorf tube and shake. Pipette again a couple of times with the tip to recover the residual extract still present in the tip. Prepare the calibration curve with PAC-A stock solution. The following final concentrations of PAC-A are used for calibration: 5-10-20-30 μg ml⁻¹. Distribute 840 μl of DMAC reagent into new Eppendorf tubes. Prepare all the reaction tubes with the DMAC solution before proceeding with the start of the colorimetric reaction. Then add 280 μl of sample to the reaction mix. Measure the absorbance at 640 nm every 30 seconds until reaching constant and light values against a blank consisting of 280 μl of extraction solvent. The calculation of the total PAC concentration expressed as PAC-A2 is obtained from the calibration curve.

The BL-DMAC method described above is used both for the determination of the total PACs of the sample to be extracted and for that of the sample after extraction.

The qualitative determination of the PACs is then carried out by means of liquid chromatography combined with mass spectrometry, but other methods as described above are also used.

The qualitative-quantitative analysis of PAC-A and PAC-B is carried out both on the sample to be extracted and on the extracted sample.

### Extraction Using Ethanol Solvent

Mix at room temperature (20-22° C.) using 96% ethanol and the plant extract until the product is homogeneous. The amount used for the example is 400 grams of dry plant extract and 1900 ml of 96% ethanol, the mixing time is 60 minutes.

The pH of the mixture is not altered.

The mixture is then filtered through a vacuum Buchner separator. The upper part is cylindrical in steel and has a flat surface containing several small holes; the filter paper is placed on this surface and made to adhere until it touches the edges. The system is connected to a vacuum pump that works at pressures of 60-120 mb.

The filtrate is then collected and concentrated using a vacuum concentrator which has an operating temperature of 60° C. and a vacuum of 60 mb. The solid unfiltered part on the filter is discarded or used for products with a lower PAC content.

The concentrated liquid is then spread on stainless steel trays and the material is dried in a static vacuum dryer, the operating temperature is 30° C. and the vacuum applied is 20 mb. Under these conditions, the time required for drying varies from 1 to 3 hours.

The dried material is then extracted from the trays and crushed with a mill. The degree of crushing may vary according to the final use of the powder. In this example, 0.5 mm sieves are used. The ground product is collected in containers with bags and then stored in a cool, dry and protected from light.

The method is then validated. In this example, samples of different plant extracts are extracted and analyzed with the BL-DMAC method and by liquid chromatography in combination with mass spectrometry.

### Results

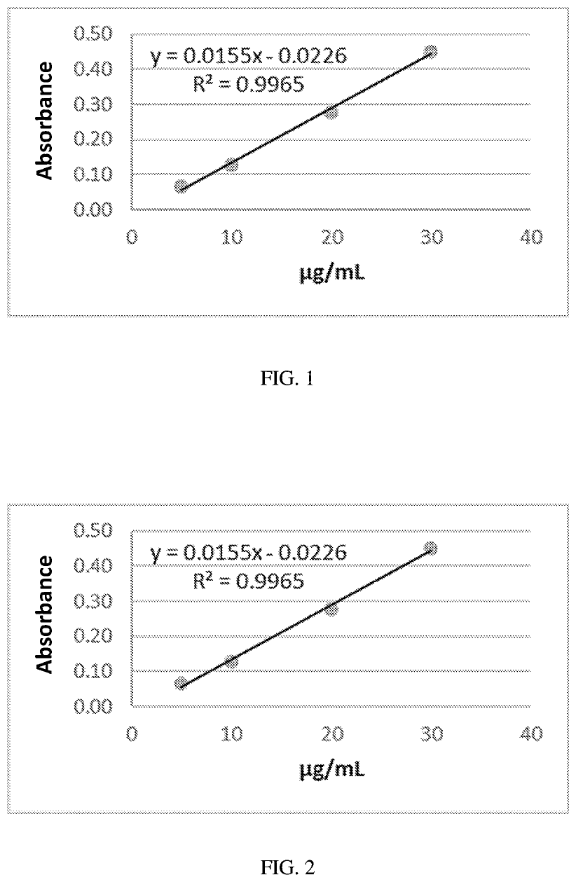

The calibration curve of the BL-DMAC method is shown in FIG. 1. The quantification leads to the following results with regard to the plant extracts before extraction:

TABLE 1
Total PAC content before extraction.

| | American | grape seed | peanut skin | pine bark | lingonberry |
|---|---|---|---|---|---|
| Specification | cranberry (mg/g) | (mg/g) | (mg/g) | (mg/g) | (mg/g) |
| Replicate 1: | 212.27 | 519.315 | 396.863 | 397.420 | 30.604 |
| Replicate 2: | 212.59 | 514.281 | 420.454 | 385.077 | 29.897 |
| Replicate 3: | 214.24 | 531.059 | 431.126 | 381.321 | 30.180 |
| Mean: | 213.03 | 521.55 | 416.15 | 387.94 | 30.23 |
| Standard Deviation | 1.06 | 8.61 | 17.53 | 8.42 | 0.36 |
| Variation Coeff. | 0.50 | 1.65 | 4.21 | 2.17 | 1.18 |

After extraction with 96% ethanol, the concentrated and pulverized filtrate shows the following values:

TABLE 2
Total PAC content after ethanolic extraction.

| | American | grape seed | peanut skin | pine bark | lingonberry |
|---|---|---|---|---|---|
| Specification | cranberry (mg/g) | (mg/g) | (mg/g) | (mg/g) | (mg/g) |
| Replicate 1: | 524.86 | 542.875 | 512.465 | 426.551 | 237.030 |
| Replicate 2: | 526.62 | 534.253 | 539.480 | 424.073 | 257.635 |
| Replicate 3: | 525.07 | 525.483 | 557.477 | 409.540 | 257.022 |
| Mean: | 525.07 | 534.204 | 536.474 | 420.055 | 250.562 |
| Standard Deviation | 1.46 | 8.696 | 22.656 | 9.190 | 11.723 |
| Variation Coeff. | 0.28 | 1.63 | 4.22 | 2.19 | 4.68 |

The above results indicate a significant increase (P<0.001) in the total PAC content. The percentage of increase of PACs is the following:

TABLE 3
Percentage of increase of PACs

| | American | grape | peanut | pine | |
|---|---|---|---|---|---|
| Specification | cranberry | seed | skin | bark | lingonberry |
| % increase | 246% | 10% | 129% | 108% | 829% |

The yields after extraction are the following:

TABLE 4
Yield of ethanolic extraction

| | American | grape | peanut | pine | |
|---|---|---|---|---|---|
| Specification | cranberry | seed | skin | bark | lingonberry |
| filtered material | 32% | 56% | 52% | 74% | 7% |
| pellet | 68% | 44% | 48% | 26% | 93% |

An analysis was also carried out on the content of PAC-A and PAC-B through HPLC-ES-MS/MS and the comparison between the raw material of the extraction is as follows:

TABLE 5
Chemical composition of PACs before extraction. Values are
expressed as percentage.

| | Molecular | American | Grape | Peanut | Pine | Lingon- |
|---|---|---|---|---|---|---|
| Polymers | Weight | cranberry | seed | skin | bark | berry |
| Flavan-3-ol | 289 | 1.18 | 24.43 | 1.28 | 3.45 | 1.25 |
| PAC A2 | 575 | 35.50 | 1.54 | 23.74 | 10.44 | 23.58 |
| PACB2 | 577 | 15.32 | 47.21 | 12.72 | 33.09 | 15.44 |
| PAC A3 | 861 | 1.31 | 0.00 | 1.10 | 0.09 | 0.10 |
| PAC AB3 | 863 | 14.30 | 0.00 | 12.84 | 6.35 | 12.50 |
| PAC B3 | 865 | 0.42 | 5.56 | 4.87 | 10.84 | 5.75 |
| PACA4 | 1147 | 0.00 | 0.00 | 1.05 | 0.00 | 0.00 |
| PAC2A1B4 | 1149 | 0.00 | 0.00 | 1.08 | 0.55 | 1.10 |
| PAC1A2B4 | 1151 | 0.02 | 0.00 | 2.92 | 0.92 | 2.46 |
| PAC B4 | 1153 | 0.00 | 0.64 | 2.24 | 2.94 | 0.89 |
| PAC3A1B5 | 1433 | 0.00 | 0.00 | 0.87 | 2.15 | 0.78 |
| PAC2A2B | 1435 | 1.37 | 0.00 | 6.59 | 5.56 | 7.41 |
| PAC1A3B5 | 1437 | 14.44 | 4.88 | 17.92 | 7.98 | 15.09 |
| PACB5 | 1439 | 12.07 | 15.43 | 8.71 | 14.89 | 10.44 |
| PAC3A2B6 | 1441 | 3.62 | 0.24 | 0.71 | 0.44 | 0.00 |
| PAC2A3B6 | 1724 | 0.01 | 0.02 | 0.31 | 0.15 | 2.90 |
| PAC1A4B6 | 1726 | 0.02 | 0.03 | 0.39 | 0.00 | 0.01 |
| PACB6 | 1728 | 0.00 | 0.02 | 0.21 | 0.08 | 0.00 |
| PAC3A3B7 | 1730 | 0.00 | 0.00 | 0.44 | 0.10 | 0.30 |
| PAC2A4B | 2012 | 0.00 | 0.00 | 0.01 | 0.00 | 0.00 |

| | | | | | |
|---|---|---|---|---|---|
| TYPE A bonds | 70.6 | 6.7 | 70.0 | 34.7 | 66.0 |
| TYPE B bonds | 27.8 | 68.9 | 30.0 | 62.8 | 34.0 |

After the extraction, the result is as follows:

TABLE 6
Chemical composition of PACs after ethanolic extraction.
Values are expressed as percentage.

| | Molecular | American | Grape | Peanut | Pine | Lingon- |
|---|---|---|---|---|---|---|
| Polymers | Weight | cranberry | seed | skin | bark | berry |
| Flavan-3-ol | 289 | 0.05 | 2.09 | 0.09 | 0.58 | 0.15 |
| PAC A2 | 575 | 51.01 | 0.72 | 7.22 | 7.74 | 11.25 |
| PACB2 | 577 | 11.13 | 79.25 | 6.34 | 30.37 | 7.32 |
| PAC A3 | 861 | 1.94 | 0.00 | 0.37 | 0.17 | 0.20 |
| PAC AB3 | 863 | 15.07 | 0.00 | 1.03 | 2.08 | 2.25 |
| PAC B3 | 865 | 0.36 | 7.47 | 6.04 | 5.85 | 4.67 |
| PACA4 | 1147 | 0.00 | 0.00 | 0.01 | 0.00 | 0.00 |
| PAC2A1B4 | 1149 | 0.01 | 0.00 | 0.04 | 0.11 | 0.14 |
| PAC1A2B4 | 1151 | 0.00 | 0.00 | 0.77 | 0.57 | 0.70 |
| PAC B4 | 1153 | 0.01 | 1.01 | 1.39 | 0.12 | 0.97 |
| PAC3A1B5 | 1433 | 0.00 | 0.00 | 0.74 | 0.00 | 0.00 |
| PAC2A2B | 1435 | 3.27 | 0.00 | 1.74 | 1.33 | 2.81 |
| PAC1A3B5 | 1437 | 15.56 | 0.00 | 18.72 | 21.34 | 41.47 |
| PACB5 | 1439 | 3.56 | 6.73 | 51.49 | 27.86 | 26.37 |
| PAC3A2B6 | 1441 | 0.00 | 0.00 | 0.15 | 0.13 | 0.00 |
| PAC2A3B6 | 1724 | 0.00 | 0.03 | 0.24 | 0.38 | 0.20 |
| PAC1A4B6 | 1726 | 0.00 | 0.00 | 0.39 | 0.00 | 0.43 |
| PACB6 | 1728 | 0.00 | 2.03 | 3.18 | 1.23 | 0.39 |
| PAC3A3B7 | 1730 | 0.00 | 0.00 | 0.03 | 0.00 | 0.15 |
| PAC2A4B | 2012 | 0.00 | 0.00 | 0.00 | 0.00 | 0.00 |

| | | | | | |
|---|---|---|---|---|---|
| TYPE A bonds | 85.0 | 1.0 | 31.0 | 34.0 | 60.0 |
| TYPE B bonds | 15.0 | 97.0 | 69.0 | 66.0 | 40.0 |

In general, extraction also affects the PAC-A percentage. In particular, an increase is found in PAC-A bonded in cranberry, whereas the PAC-A percentage of lingonberry and pine bark is not affected. An increase in PAC B is found in grape seeds and peanut skin.

In general, therefore, the disclosed extraction process leads to an increase of the total content of PACs and an increase of the total content of PAC-A in some plant species.

### Example 2. Increasing the PAC Content of a Plant Extract Containing PACs by Using Acetone as a Solvent

### Materials and Methods

A method for increasing the concentration of total PAC and the percentage of PAC-A (with particular reference to dimers and trimers) in commercial samples with a PAC content is described below.

Increased total PAC content starting from American cranberry, grape seed, peanut skin, pine bark and lingonberry extracts.

Before proceeding, the chemical characterization of the extracts is performed through assays on the total content of PAC (BL-DMAC assay) and the chemical characterization of PAC-A is done by liquid chromatography combined with mass spectrometry as described in EXAMPLE 1.

The qualitative determination of the PACs is then carried out by means of liquid chromatography combined with mass spectrometry, but other methods as described in EXAMPLE 1 are also used.

The qualitative-quantitative analysis of PAC-A and PAC-B is carried out both on the sample to be extracted and on the extracted sample as described in EXAMPLE 1.

### Results

The calibration curve of the BL-DMAC method is shown in FIG. 2. The quantification leads to the following results with regard to the plant extracts before extraction:

TABLE 7
Total PAC content before extraction.

| | grape seed | peanut skin | pine bark | lingonberry |
|---|---|---|---|---|
| Specification | (mg/g) | (mg/g) | (mg/g) | (mg/g) |
| Replicate 1: | 519.315 | 396.863 | 397.420 | 30.604 |
| Replicate 2: | 514.281 | 420.454 | 385.077 | 29.897 |
| Replicate 3: | 531.059 | 431.126 | 381.321 | 30.180 |
| Mean: | 521.55 | 416.15 | 387.94 | 30.23 |
| Standard Deviation | 8.61 | 17.53 | 8.42 | 0.36 |
| Variation Coeff. | 1.65 | 4.21 | 2.17 | 1.18 |

After extraction with 100% acetone, the concentrated and pulverized filtrate shows the following values:

TABLE 8
Total PAC content after acetone extraction

| | grape seed | peanut skin | pine bark | lingonberry |
|---|---|---|---|---|
| Specification | (mg/g) | (mg/g) | (mg/g) | (mg/g) |
| Replicate 1: | 590.778 | 560.689 | 454.838 | 238.497 |
| Replicate 2: | 591.982 | 561.183 | 434.262 | 268.593 |
| Replicate 3: | 598.731 | 570.988 | 440.243 | 267.160 |
| Mean: | 593.83 | 564.29 | 443.11 | 258.08 |
| Standard Deviation | 4.29 | 5.81 | 10.58 | 16.98 |
| Variation Coeff. | 0.72 | 1.03 | 2.39 | 6.58 |

The above results indicate a significant increase (P<0.001) in the total PAC content. The percentage of increase of PACs is the following:

TABLE 9
Percentage of increase of PACs

| Specification | grape seed | peanut skin | pine bark | lingonberry |
|---|---|---|---|---|
| % increase | 113% | 136% | 114% | 853% |

The yields after extraction are the following:

TABLE 10
Yield of acetone extraction

| Specification | grape seed | peanut skin | pine bark | lingonberry |
|---|---|---|---|---|
| filtered material | 56.07% | 49.91% | 76.43% | 7.71% |
| pellet | 43.93% | 50.09% | 23.57% | 92.29% |

An analysis was also carried out on the content of PAC-A and PAC-B through HPLC-ESI-MS/MS and the comparison between the raw material of the extraction is as follows:

TABLE 11
Chemical composition of PACs before extraction.
Values are expressed as percentage.

| | Molecular | Grape | Peanut | Pine | |
|---|---|---|---|---|---|
| Polymers | Weight | seed | skin | bark | Lingonberry |
| Flavan-3-ol | 289 | 24.43 | 1.28 | 3.45 | 1.25 |
| PAC A2 | 575 | 1.54 | 23.74 | 10.44 | 23.58 |
| PACB2 | 577 | 47.21 | 12.72 | 33.09 | 15.44 |
| PAC A3 | 861 | 0.00 | 1.10 | 0.09 | 0.10 |
| PAC AB3 | 863 | 0.00 | 12.84 | 6.35 | 12.50 |
| PAC B3 | 865 | 5.56 | 4.87 | 10.84 | 5.75 |
| PACA4 | 1147 | 0.00 | 1.05 | 0.00 | 0.00 |
| PAC2A1B4 | 1149 | 0.00 | 1.08 | 0.55 | 1.10 |
| PAC1A2B4 | 1151 | 0.00 | 2.92 | 0.92 | 2.46 |
| PAC B4 | 1153 | 0.64 | 2.24 | 2.94 | 0.89 |
| PAC3A1B5 | 1433 | 0.00 | 0.87 | 2.15 | 0.78 |
| PAC2A2B | 1435 | 0.00 | 6.59 | 5.56 | 7.41 |
| PAC1A3B5 | 1437 | 4.88 | 17.92 | 7.98 | 15.09 |
| PACB5 | 1439 | 15.43 | 8.71 | 14.89 | 10.44 |
| PAC3A2B6 | 1441 | 0.24 | 0.71 | 0.44 | 0.00 |
| PAC2A3B6 | 1724 | 0.02 | 0.31 | 0.15 | 2.90 |
| PAC1A4B6 | 1726 | 0.03 | 0.39 | 0.00 | 0.01 |
| PACB6 | 1728 | 0.02 | 0.21 | 0.08 | 0.00 |
| PAC3A3B7 | 1730 | 0.00 | 0.44 | 0.10 | 0.30 |
| PAC2A4B | 2012 | 0.00 | 0.01 | 0.00 | 0.00 |

| | | | | |
|---|---|---|---|---|
| TYPE A bonds | 6.7 | 70.0 | 34.7 | 66.0 |
| TYPE B bonds | 68.9 | 30.0 | 62.8 | 34.0 |

After the extraction, the result is as follows:

TABLE 12
Chemical composition of PACs after acetone extraction.
Values are expressed as percentage.

| | Molecular | Grape | Peanut | Pine | |
|---|---|---|---|---|---|
| Polymers | Weight | seed | skin | bark | Lingonberry |
| Flavan-3-ol | 289 | 0.92 | 0.39 | 0.40 | 0.28 |
| PAC A2 | 575 | 1.86 | 10.89 | 5.13 | 17.54 |
| PACB2 | 577 | 39.80 | 4.01 | 5.02 | 2.31 |
| PAC A3 | 861 | 0.01 | 1.60 | 0.07 | 0.07 |
| PAC AB3 | 863 | 0.06 | 3.73 | 1.38 | 3.09 |
| PAC B3 | 865 | 5.74 | 1.15 | 0.74 | 0.37 |
| PACA4 | 1147 | 0.44 | 0.01 | 0.50 | 0.22 |
| PAC2A1B4 | 1149 | 0.53 | 0.47 | 0.34 | 0.66 |
| PAC1A2B4 | 1151 | 0.53 | 1.12 | 0.26 | 0.52 |
| PAC B4 | 1153 | 1.16 | 0.20 | 0.09 | 0.02 |
| PAC3A1B5 | 1433 | 0.04 | 1.13 | 2.56 | 2.61 |
| PAC2A2B | 1435 | 0.01 | 1.96 | 28.00 | 23.07 |
| PAC1A3B5 | 1437 | 0.24 | 57.73 | 27.38 | 39.15 |
| PACB5 | 1439 | 41.31 | 10.45 | 24.00 | 6.19 |
| PAC3A2B6 | 1441 | 4.54 | 1.12 | 0.85 | 0.79 |
| PAC2A3B6 | 1724 | 0.45 | 1.16 | 1.90 | 1.76 |
| PAC1A4B6 | 1726 | 0.02 | 1.74 | 0.90 | 0.80 |
| PACB6 | 1728 | 1.87 | 0.45 | 0.11 | 0.01 |
| PAC3A3B7 | 1730 | 0.04 | 0.54 | 0.31 | 0.49 |
| PAC2A4B | 2012 | 0.43 | 0.14 | 0.05 | 0.06 |

| | | | | |
|---|---|---|---|---|
| TYPE A bonds percentage | 7.46 | 17.73 | 37.76 | 45.44 |
| TYPE B bonds percentage | 54.92 | 16.84 | 29.55 | 11.03 |

In general, the disclosed extraction increases the total PAC content without altering the PAC-A percentage.

### Example 3. A Formulation Based on Plant Extracts with Increased Total PAC Content

### Materials and Methods

The plant extracts with an increased PAC content described in Examples 1 and 2, or simply the combination of extracts, have been used to prepare a formulation aimed to obtain a calibrated content of PACs to be used for the evaluation of the antibacterial and antiviral activity.

The formulation is based on the PAC content of the individual plant extracts. Grape seed, peanut skin, pine bark and lingonberry extracts with increased PAC-A content are used to prepare the formulation.

Based on the total PAC and A-type PAC contents as described in Examples 1 and 2 the formulation considers the following percentages: Grape seed extract before and after ethanol or acetone extraction in percentages ranging from 1 to 25%, preferably between 1 and 9%, more preferably 2%.

Peanut skin extract before and after ethanol or acetone extraction in percentages ranging from 40 to 85%, preferably between 50 and 75%, more preferably 68%.

Pine bark extract before and after ethanol or acetone extraction in percentages ranging from 1 to 35%, preferably between 5 and 20%, more preferably 10%.

Lingonberry extract before and after ethanol or acetone extraction in percentages ranging from 5 to 45%, preferably between 10 and 32%, more preferably 20%. Table 13. PAC content of the formulation (indicated here as SP4), before extraction (SP4), after extraction with ethanol (SP4E) and after extraction with acetone (SP4A)

| | | | | | | | | | Total | Total |
|---|---|---|---|---|---|---|---|---|---|---|
| | | | | | | | | | PAC | PAC |
| | | | | | | Total | Total | | of | of |
| | | Total | Total | | Total | PAC | PAC | Total | formulation | formulation |
| | Total | PAC | PAC | | PAC | expected | expected | PAC | after | after |
| | PAC | content | content | | expected | after | after | of | ethane | acetone |
| | content | after | after | Formulation | before | ethanol | acetone | formulation | extraction | extraction |
| | before | ethanol | acetone | or SP4 | extraction | extraction | extraction | before | or | or |
| | extraction | extraction | extraction | (percentage | of | or | or | extraction | SP4E | SP4A |
| Plant | (DMAC, | (DMAC, | (DMAC, | of each | SP4 | SP4E | SP4A | (DMAC, | (DMAC, | (DMAC, |
| extracts | mg/g) | mg/g) | mg/g) | extract) | (mg/g) | (mg/g) | (mg/g) | mg/g) | mg/g) | mg/g) |
| Grape | 522 | 534 | 594 | 2% | 10 | 11 | 12 | 379 | 524 | 548 |
| seed | | | | | | | | (±2.6) | (±9.1) | (±0.8) |
| Peanut | 416 | 536 | 564 | 68% | 283 | 365 | 384 | | | |
| skin | | | | | | | | | | |
| Pine | 388 | 420 | 443 | 10% | 39 | 42 | 44 | | | |
| bark | | | | | | | | | | |
| Lingonberry | 30 | 251 | 258 | 20% | 6 | 50 | 52 | | | |
| TOTAL | | | | 100% | 338 | 468 | 492 | | | |

Therefore, the formulation contains more than 50% total PACs.

The yields after extraction are the following:

TABLE 14
Yield of total PACs.

| | Formulation after | Formulation after |
|---|---|---|
| | ethanol extraction | acetone extraction |
| Specification | or SP4E | or SP4A |
| filtered material | 31% | 43% |
| pellet | 68% | 57% |

The tapped bulk density of the formulation is 542.86 g/L (or 543 kg/m³).

An analysis is also carried out on the content of PAC-A and PAC-B through HPLC-ESI-MS/MS and the comparison between the formulation before and after extraction is as follows:

TABLE 15
Chemical composition of PACs in the formulation before
(SP4) and after extraction with ethanol (SP4E) and
acetone (SP4A). Values are expressed as percentage.

| | | Formulation | Formulation | Formulation |
|---|---|---|---|---|
| | | before | after ethanol | after acetone |
| | Molecular | extraction of | extraction or | extraction or |
| Polymers | Weight | SP4 | SP4E | SP4A |
| Flavan-3-ol | 289 | 0.11 | 0.09 | 0.54 |
| PAC A2 | 575 | 17.68 | 16.35 | 16.88 |
| PACB2 | 577 | 16.00 | 18.55 | 9.83 |
| PAC A3 | 861 | 0.22 | 0.44 | 0.51 |
| PAC AB3 | 863 | 3.90 | 3.48 | 5.03 |
| PAC B3 | 865 | 9.66 | 2.79 | 2.58 |
| PACA4 | 1147 | 7.19 | 1.97 | 0.03 |
| PAC2A1B4 | 1149 | 0.25 | 0.30 | 1.19 |
| PAC1A2B4 | 1151 | 0.93 | 0.91 | 2.11 |
| PAC B4 | 1153 | 3.19 | 3.46 | 0.14 |
| PAC3A1B5 | 1433 | 0.50 | 0.98 | 2.63 |
| PAC2A2B | 1435 | 7.47 | 10.36 | 14.02 |
| PAC1A3B5 | 1437 | 7.19 | 9.68 | 13.78 |
| PACB5 | 1439 | 13.92 | 23.29 | 20.71 |
| PAC3A2B6 | 1441 | 0.79 | 1.32 | 2.92 |
| PAC2A3B6 | 1724 | 1.98 | 2.76 | 1.70 |
| PAC1A4B6 | 1726 | 2.32 | 1.85 | 3.60 |
| PACB6 | 1728 | 5.82 | 0.81 | 0.38 |
| PAC3A3B7 | 1730 | 0.35 | 0.56 | 1.17 |
| PAC2A4B | 2012 | 0.53 | 0.05 | 0.25 |

| | | | |
|---|---|---|---|
| TYPE A bonds | 51.3 | 51.0 | 68.5 |
| TYPE B bonds | 49.5 | 49.8 | 31.5 |

The total PAC-A content is not affected by the ethanol extraction and is increased after acetone extraction.

In summary, the formulation contains a high total PAC content (>50%) and a 50% percentage of PAC-As. The yield is more than 30% with ethanol extraction and reaches 43% with acetone extraction.

### Example 4. Fractionation and purification of the formulation SP4

### Materials and Methods

The formulation described in Example 3 is used to purify the different fractions.

In order to separate the different classes of molecule contained in the formulation, Sephadex® LH20, a liquid chromatography media for molecular sizing of natural product is used. Sephadex® LH20 is useful for analytical and industrial scale for preparation of closely related molecular species and has been specifically developed for gel filtration of natural products, such as polyphenols dissolved in organic solvents.

Sephadex® LH-20, due to its physical-chemical properties, can be used either during initial purification prior to polishing by high performance ion exchange or reversed phase chromatography or as the final polishing steps. Sephadex® LH-20 is characterized by chromatographic selectivity due to dual hydrophilic and lipophilic nature of the matrix, easily predicted elution behavior based on the chemical structure 1-3, 16 of the sample, chemical and physical robustness and excellent batch to batch reproducibility.

Sephadex® LH-20 is first poured into a backer containing 70% Ethanol by using 3.6 to 3.9 ml of 70% Ethanol per gram of Sephadex® LH-20, for at least 3 h at room temperature in the solvent. A preparative solvent resistant glass column (about 40 cm in height by 2 cm in diameter) is filled by ⅓ with 70% Ethanol and then the Sephadex® LH-20 dissolved in 70% Ethanol is poured into the column. The same procedure can be used for steel columns of higher volume, with proportional volumes of liquids and resin, e.g. for scaling up the process. The volume of the column is calculated by multiplying the height of the column by the area of its cross section (πr²×h) and Sephadex® LH-20 is packed at 300 ml/h with 70% Ethanol until the bed has reached a constant height. The column is equilibrated with at least 2 column volumes of 70% Ethanol. The flow is then stopped by means of a valve placed at the bottom of the column. The volume of the packed Sephadex® LH-20 is calculated as the volume of the column by considering the height of the Sephadex® LH-20 packed column.

The formulation SP4 is then dissolved in 70% Ethanol by using 0.2 g of SP4 for every ml of 70% Ethanol (20% solution). The sample solution is settled and any fine particles of medium is decanted. The sample solution is then resuspended and poured into the column in one continuous step (using a glass rod to help to eliminate air bubbles). The sample is then adsorbed into the Sephadex® LH-20 until the sample has reached the top by opening the valve placed at the bottom of the column. The valve is then closed and the top of the Sephadex® LH-20 is carefully filled with 70% Ethanol. The top of the column is then closed with a lid and then connected to a pump avoiding the presence of air bubbles. The bottom valve is opened and the pump starts flowing 70% Ethanol from a solvent reservoir. The bottom valve is connected by a solvent resistant tube to a fraction collector. The pump is set to 300 ml/h and fractions of the eluate from the column are collected.

After 2 column volumes of the 70% Ethanol elution (Elution A) the pump is stopped and the bottom valve closed. The pump is then connected to a solvent reservoir that contains a mixture of 96% Ethanol, 100% Methanol and water in the following proportions 40:40:20. The bottom valve is then opened, the pump is set to 300 ml/h and fractions of the eluate from the column are collected.

After 2 column volumes of the 96% Ethanol, 100% Methanol and water elution (Elution B) the pump is stopped and the bottom valve closed. The pump is then connected to a solvent reservoir that contains Acetone and water with the following proportions, 80:20. The bottom valve is then opened, the pump is set to 300 ml/h and fractions of the eluate from the column are collected.

After 2 column volumes of the 80% Acetone/water elution (Elution C) the pump is stopped and the bottom valve closed. The pump is then connected to a solvent reservoir that contains 10% Acetone. The bottom valve is then opened, the pump is set to 300 ml/h and fractions of the eluate from the column are collected.

After 2 column volumes of the 100% Acetone elution (Elution D) the pump is stopped and the bottom valve closed. The pump is then connected to a solvent reservoir that contains 70% Ethanol and the column is the conditioned with 3 column volumes and is prepared for the next separation.

Fractions from Elution A, B, C and D are individually analyzed in order to evaluate the content of PACs by using the methods described in Example 1 (DMAC and HPLC analyses). An eluogram from Sephadex-LH20 fractionation is obtained by reading each individual fraction with a spectrophotometer at the wavelength of 360 nm and by the DMAC assay at 640 nm (as explained in Example 1).

Fractions containing the highest amount of PACs according to the DMAC assay are pooled and dried under vacuum for chemical analysis. The chemical characterization of the fractions is performed through assays on the total content of PAC (BL-DMAC assay) and the chemical characterization of PAC-A is done by liquid chromatography combined with mass spectrometry as described in EXAMPLE 1.

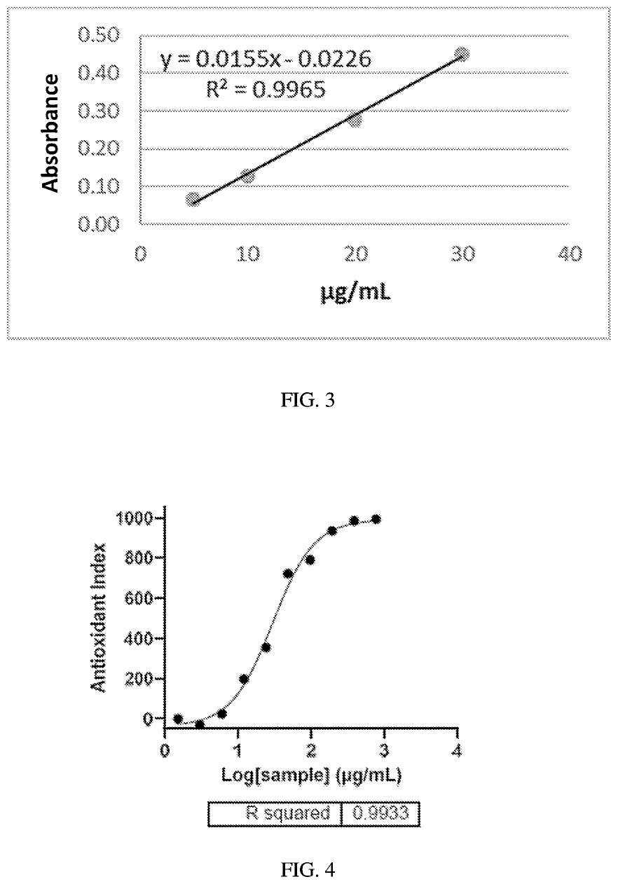

The qualitative determination of the PACs is then carried out by means of liquid chromatography combined with mass spectrometry, but other methods as described in EXAMPLE 1 are also used. The qualitative-quantitative analysis of PAC-A and PAC-B is carried out both on the sample to be extracted and on the extracted sample as described in EXAMPLE 1. The calibration curve of the BL-DMAC method is shown in FIG. 3. The quantification leads to the following results with regard to the fractions collected:

TABLE 16
Total PAC content of the different elutions.

| | | | Pooled C and |
|---|---|---|---|
| | Elution A | Elution B | D elutions |
| Specification | (mg/g) | (mg/g) | (mg/g) |
| Replicate 1: | ND | ND | 780.54 |
| Replicate 2: | ND | 0.52 | 807.03 |
| Replicate 3: | ND | 0.51 | 795.56 |
| Mean: | ND | 0.52 | 794.38 |
| Standard Deviation | NC | 0.003 | 13.29 |
| Variation Coeff. | NC | 0.5% | 1.67% |

ND = not detectable; NC = not computable.
Fractions C and D possess the bulk of total PACs.

An analysis was also carried out on the content of PAC-A and PAC-B through HPLC-ESI-MS/MS as well as for other molecules.

TABLE 17
Chemical composition of PACs in the different
elutions. Values are expressed as percentage.

| | | | | Pooled C and |
|---|---|---|---|---|
| | Molecular | Elution A | Elution B | D elutions |
| Molecule | Weight | (mg/g) | (mg/g) | (mg/g) |
| Flavan-3-ol | 289 | ND | ND | 2.04 |
| PAC A2 | 575 | ND | ND | 18.11 |
| PACB2 | 577 | ND | ND | 11.63 |
| PAC A3 | 861 | ND | ND | 4.69 |
| PAC AB3 | 863 | ND | ND | 3.71 |
| PAC B3 | 865 | ND | ND | 2.59 |
| PACA4 | 1147 | ND | ND | 1.32 |
| PAC2A1B4 | 1149 | ND | ND | 2.94 |
| PAC1A2B4 | 1151 | ND | ND | 7.06 |
| PAC B4 | 1153 | ND | ND | 2.60 |
| PAC3A1B5 | 1433 | ND | ND | 1.34 |
| PAC2A2B | 1435 | ND | ND | 5.19 |
| PAC1A3B5 | 1437 | ND | ND | 8.52 |
| PACB5 | 1439 | ND | ND | 19.92 |
| PAC3A2B6 | 1441 | ND | ND | 1.32 |
| PAC2A3B6 | 1724 | ND | ND | 1.33 |
| PAC1A4B6 | 1726 | ND | ND | 2.69 |
| PACB6 | 1728 | ND | ND | 0.37 |
| PAC3A3B7 | 1730 | ND | ND | 0.69 |
| PAC2A4B | 2012 | ND | ND | 1.95 |

| | | | |
|---|---|---|---|
| TYPE A bonds | NC | NC | 35.59 |
| TYPE B bonds | NC | NC | 64.41 |

ND = not detectable;
NC = not computable

Fractions C and D contain the bulk of PAC-A, which are practically absent in the other two fractions. By considering the high content of total PACs (794.38 mg/g) of fractions C-D the total amount of PAC-A of fractions C-D is 282.76 mg/g).

### Example 5. Determination of the Allergen Content of Formulation SP4

Formulation SP4 contains a consistent percentage of peanut skin extract. Therefore it is important to evaluate the possible presence of allergens. To evaluate the content of peanut allergens in the formulation SP4 two analytical methods were used: ELISA and DNA methods.

ELISA—Enzyme Immunoassay for the Quantitative Analysis of Peanuts Allergens Materials and Methods This is a sandwich enzyme immunoassay for the quantitative analysis of peanuts in food. The basis of the test is an antigen-antibody reaction. The wells of the microplate are sensitized with specific antibodies to peanut proteins. When standards or sample solutions are added to the wells, the peanut proteins bind to the specific capture antibodies, giving rise to an antigen antibody complex. The components of the sample not bound by the antibodies are then washed away. The antibody conjugated with peroxidase is then added which binds to the antigen-antibody complex forming the antibody-antigen-antibody sandwich complex. The unbound conjugated enzyme is eliminated with a wash. The conjugated enzyme converts the chromogen into a blue product. The addition of the stop solution causes a color change from blue to yellow. Quantitative determination is performed photometrically at 450 nm. The absorbance value is proportional to the concentration of the peanut protein contained in the sample. The result is expressed in mg/kg (ppm) of peanut. It is possible to determine the conversion factor (starting from the protein content of the standard material used) in order to calculate the concentration of peanut proteins per kg of food. The conversion factor is 0.222. Multiply the result expressed in mg of peanut per kg of food with this factor.

### Results

After analyzing the formulation, the result obtained is <2.5 ppm (mg/kg), a value well below the threshold limits (1.5 mg of protein/dose). Considering that 2.5 mg are present in one kg of the formulation and that the formulation is used at a maximum dosage of 600 mg (see EXAMPLE 9), the dose of allergens in 600 mg product is about 1.5 μg, that is 1,000 times lower than the suggested threshold limits.

DNA—Real-Time PCR for the Direct, Qualitative Detection and Differentiation of Specific Peanut (Arachis hypogaea) DNA Sequences According to Directive (EC) 1169/2011.

### Materials and Methods

The PCR method has a limit of detection of ≤1 mg/kg (ppm), because PCR systems are very sensitive even a small amount of target DNA is sufficient for a successful analysis. We used the SureFood® ALLERGEN 4plex Peanut, a real-time PCR for the direct, qualitative detection and differentiation of specific Peanut (Arachis hypogaea) DNA sequences according to directive (EC) 1169/2011.

Each reaction contains an internal amplification control (IAC). If the DNA contains PCR inhibiting substances, the signal of the amplification control will be affected or the amplification will be suppressed. The real-time PCR assay can be performed with commonly used real-time PCR instruments, equipped for detection of four fluorescence emissions at the channels FAM at the same time. The evaluation is made according to the usual analysis program recommended by the real-time PCR instrument manufacturer.

A sample is stated positive for the respective parameter, if the sample DNA shows amplification in the respective channel. High amplicon concentrations can result in a weak or absent signal of the internal amplification control (IAC). A sample is stated negative for the respective parameter, if the sample DNA shows no amplification in the respective channel compared to the negative control.

### Results

After analyzing the formulation, the result obtained is <1.0 ppm (mg/kg), a value well below the threshold limits (1.5 mg of protein/dose). Considering that this more sensitive test shows that 1.0 mg are present in one kg of the formulation and that the formulation is used at a maximum dosage of 600 mg (see EXAMPLE 9), the dose of allergens in 600 mg product is about 0.6 μg, that is about 2,500 times lower than the suggested threshold limits.

Taken together, the two methods demonstrate that the allergen count of the formulation SP4 is a hundred times below the threshold limits and that the product is safe also for allergic persons.

### Example 6. Intracellular antioxidant activity of the formulation SP4

### Materials and Methods

To evaluate the antioxidant activity of the formulation SP4 of Example 3, we used the Anti Oxidant Power 1 (AOP1) assay which allows the evaluation of antioxidant activity by direct measurement of neutralization of intracellular free radicals.

The assay relies on the controlled generation of intracellular radical species by a photo-induction process. A cell permeant biosensor, thiazole orange (TO), is added to the cell culture medium and binds to nucleic acids with a low fluorescence level. When TO is photoactivated by appropriate LED illumination, its relaxation is accompanied by an energy transfer to the intracellular dioxygen molecule (³O₂) resulting in the production of singlet oxygen (¹O₂) which in turn triggers a cascade of Reactive Oxygen Species (ROS) production including the free radical species superoxide anion (O₂—) and hydroxyl radical (OH—). Presence of ROS species leads to cell alteration and fluorescence level increase. Neutralization of intracellular free radicals by sample added in the culture medium inhibits this process, maintaining fluorescence emission at low level. Kinetic records allow for antioxidant index calculation. Dose-response curves fitting with sigmoid model allow for efficacy standard concentrations (EC₁₀, EC₅₀, EC₉₀) evaluation. Detection is done by fluorescence (exc/em 505-535).

The study was performed on human HepG2 cells. Cells were seeded in 96-well plates at a density of 75000 cells/well in DMEM medium supplemented with Fetal Calf Serum (FCS) and kept in the incubator for 24 h at 37° C./5% CO₂.

For AOP1, cells were incubated in the presence of FP4 (8 concentrations obtained by serial log 2 dilutions) during 4 h (AOP1) at 37° C./5% CO₂. Experiments were carried out in DMEM medium without FCS.

For AOP1, cells were treated after the 4 h incubation with the fluorescent biosensor (TO=thiazole orange) during 1 h and fluorescence was measured (RFU at 535 nm) according to a recurrent 480 nm LED application procedure (20 iterations) of the whole 96-well plate. Kinetic profiles were recorded. Normalized data (percentage of control values) are also obtained.

### Results

The dose-response curves is presented in FIG. 4. Antioxidant cell index (AOP index) is calculated from normalized kinetic profiles according to the formula:

AOP index (%)=100−100 (₀∫²⁰ RFU_{SP4}/₀∫²⁰ RFU_{control})

Dose-response curves, obtained by compiling AOP indices according to logarithm(10) of the sample concentration, are submitted to a sigmoid fit according to the formula:

AOP index=AOP indexₘᵢₙ+(AOP indexₘₐₓ−AOP indexₘᵢₙ)/(1+10^{(Log(EC50-SC)*HS)})

where SC═SP4 concentration and HS=Hill slope. EC₅₀ (50% efficacy concentration), EC₁₀ and EC₉₀ are then evaluated according to the fit.

The formulation SP4 showed an antioxidant effect in human HepG2 liver cells. SP4 demonstrates a direct antioxidant activity with a maximum effect for intracellular free radical quenching with an AOP1 EC₅₀ of 30.11 μg/ml.

A direct comparison of the obtained data with data from the literature (dx.doi.org/10.3390/antiox9060471) indicates that SP4, with an EC₅₀ of 30.11 μg/ml, has similar antioxidant activity of BHA (31.54 μg/ml) and BHT (34.25 μg/ml), twice as much the antioxidant activity of resveratrol (64.66 μg/ml), 4.5 times that of Trolox (138.50 μg/ml) and 20.7 times the antioxidant activity of epicatechin.

### Example 7. In Vitro Efficacy of the Formulation SP4 to Inhibit Different Respiratory Viruses

### Influenza Virus

Cells, culture conditions and viruses: The Madin-Darby canine kidney (MDCK) (ATCC CCL-34) and the human adenocarcinoma alveolar basal epithelial A549 (ATCC CCL-185) cell lines were purchased from ATCC and propagated in DMEM supplemented with 10% fetal bovine serum (FBS; Euroclone), 2 mM L-glutamine, 1 mM sodium pyruvate, 100 U/ml penicillin, and 100 μg/ml streptomycin sulfate. Infections were performed in the presence of 1 μg/ml of trypsin TPCK treated from bovine pancreas (Sigma-Aldrich) and 0.14% of Bovine Serum Albumin (Sigma-Aldrich).

The influenza virus strains A/Puerto Rico/8/34 (IAV) (VR-1469) and B B/Lee/40 (IBV) (VR-101) were obtained from ATCC. IAV and IBV were cultured and titrated by plaque assay on MDCK cells as described by Luganini et al., Front. Microbiol. 9:1826, 2018. Antiviral Assays—Cytoxicity of SP4 was determined on A549 cells after a 72 h of treatment by means of the Cell Titer Glo(R) Luminescent Cell Viability assay (Promega).

The antiviral activity of SP4 was determined by Virus Yield Reduction Assay (VRA). To this end, A549 cells were seeded in 24-well plates (3×105 cells/well) and after 24 h they were exposed 1 h prior to infection to increasing concentrations of SP4 and then infected with IAV or IBV (40 PFU/well). After virus adsorption (1 h at 37° C.), cultures were incubated in presence of SP4 for 48 h. Thereafter, infectious IAV or IBV in supernatants was titrated by plaque assay in MDCK cells (seeded in 48-well plates at 1.2×105 cell/well). After virus adsorption (1 h at 37° C.), cultures were incubated in medium containing 0.7% Avicel (FMC BioPolymer) plus MEDS433 or Brequinar. At 48 h post-infection (h p.i.), the cells were fixed with a solution of 4% formaldehyde in phosphate-buffered saline 1×(PBS) for 1 h at room temperature (RT) and stained with a solution of 1% crystal violet. The microscopic plaques count then allowed to define the concentration of either MEDS433 produced 50 reduction in plaque formation (EC50) (Luganini et al., Front. Microbiol. 9:1826, 2018).

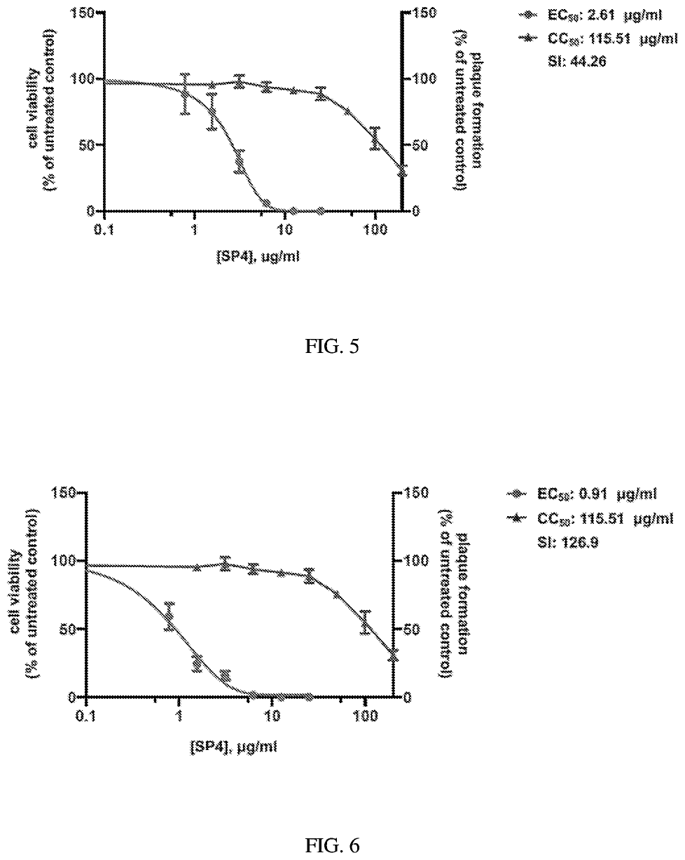

It was demonstrated that SP4 inhibits IAV replication (FIG. 5) and IBV replication (FIG. 6).

### Respiratory Syncytial Virus (RSV)

Cells, culture conditions and viruses: A549 and HEp-2 cells (ATCC CCL-23) were purchased from ATCC and maintained in Dulbecco's Modified Eagle Medium (DMEM; Euroclone) supplemented with 10% fetal bovine serum (FBS; Euroclone), 2 mM L-glutamine, 1 mM sodium pyruvate, 100 U/ml penicillin, and 100 mg/ml streptomycin sulfate.

The Respiratory Syncytial Virus (RSV) strains A-Long (VR-26) was obtained from ATCC and propagated and titrated on HEp-2 cells as described by Rameix-Welti et al. Nat. Commun. 5:5104, 2014.

Antiviral Assays: Cytoxicity assays of SP4 were performed on A549 cells with the Cell Titer Glo(R) Luminescent Cell Viability assay (Promega) after 72 h of incubation with the compound.

The antiviral activity of SP4 was determined by VRA. Briefly, A549 cells were seeded in 24-well plates (3×105 cells/well) and after 24 h they were treated with different concentrations of SP4 1 h prior to infection, and then infected with RSV A (50 PFU/well). Following virus adsorption (2 h at 37 C), cultures were maintained in medium-containing SP4. At 72 h post infection (h.p.i.), infectious RSV A in cell supernatants was titrated by plaque assay in HEP-2 cells (seeded in 48-well plates at 1.2×105 cell/well). Following virus adsorption (2 h at 37 C), cultures were maintained in medium-containing 0.3% methylcellulose (Sigma) plus compounds. At 96 h post infection (h.p.i.), cells were fixed and stained by using 20% ethanol and 1% crystal violet. Plaques were microscopically counted, and the mean plaque counts for each concentration expressed as a percentage of the mean plaque count for the control virus. The plaque numbers were plotted against the compound concentrations and the EC50 was determined as the compound concentration producing 50% reduction of the plaque numbers.

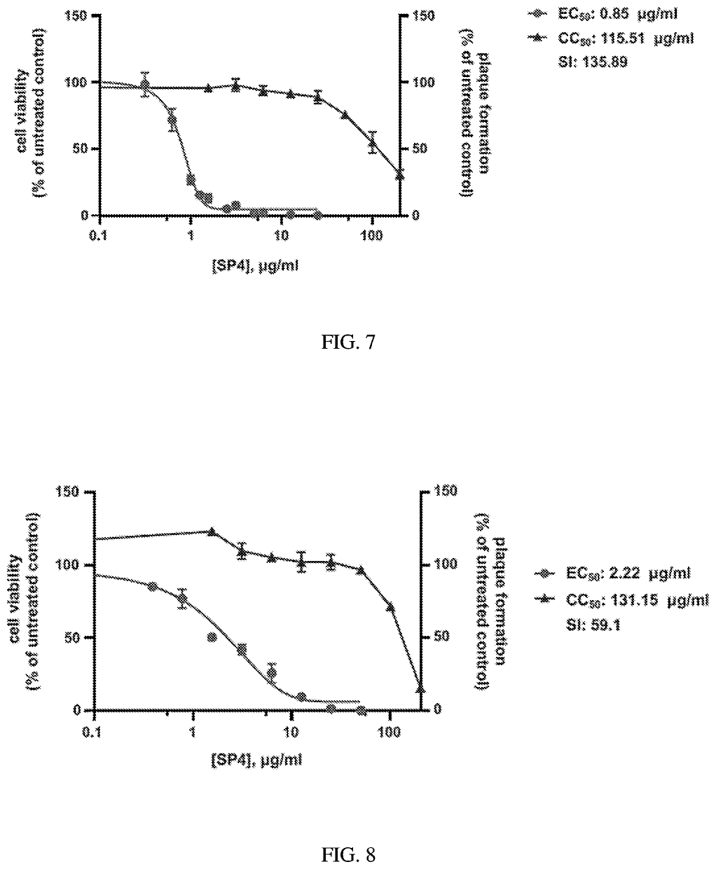

It was demonstrated that SP4 inhibits RSV replication (FIG. 7).

hCoV-OC43

Cells, culture conditions and viruses: The human colorectal carcinoma HCT-8 (ATCC CCL-244) and the human lung adenocarcinoma Calu-3 (ATCC HTB-55) cells were purchased from ATCC and maintained in Dulbecco's Modified Eagle Medium (DMEM) supplemented with 10% fetal bovine serum (FBS), 2 mM L-glutamine, 1 mM sodium pyruvate, 100 U/ml penicillin, and 100 mg/ml streptomycin sulfate.

hCoV-OC43 (ATCC VR-1558) was obtained from ATCC, and propagated and titrated in HCT-8 cells as described by Calistri, Luganini et al., Microorganisms 9, 173, 2021.

Antiviral assay: Cytoxicity assays of SP4 was performed on HCT-8 cells by means of the Cell Titer Glo(R) Luminescent Cell Viability assay (Promega) after 72 h of incubation with the compound.

The antiviral activity of SP4 was determined by Focus Forming Reduction Assay (FFRA) as previously described (Calistri, Luganini et al., Microorganisms 9, 173, 2021). Briefly, HCT-8 cell monolayers were treated with with SP4 1 h prior to and during infection with the hCoV-OC43 (100 PFU/well). At 72 h post-infection (p.i.), cell monolayers were fixed, and subjected to indirect immunoperoxidase staining with a mAb against the hCoV-OC43 N protein (clone.542-D7; Millipore, Burlington, MA, USA) (diluted 1:100). Viral foci were microscopically counted, and the mean counts for each drug concentration were expressed as a percentage of the mean plaque counts of control virus. Then the EC50 was determined as the compound concentration producing 50% reduction of hCoV-OC43 infectivity.

It was demonstrated that SP4 inhibits hCoV-OC43 replication (FIG. 8).

Taken together, these results demonstrate that formulation SP4 is a potent antiviral agent against respiratory viruses.

### Example 8. In Vitro Bacterial Antiadhesion Activity of the Formulation SP4 Utilizing an HRBC Hemagglutination Assay Specific for Uropathogenic P-Fimbriated Escherichia coli

### Materials and Methods

The formulation SP4 was tested for in vitro bacterial anti-adhesion activity (AAA) on a per weight basis. The formulation SP4 was suspended (60 mg/ml) in PBS, neutralized with 1 N NaOH, diluted serially (2-fold), and tested for bacterial antiadhesion activity utilizing an HRBC hemagglutination assay specific for uropathogenic P-fimbriated E. coli. The concentration at which hemagglutination activity was suppressed by 50% was recorded as an indicator of the strength of the bacterial anti-adhesion activity (AAA). Antiadhesion assays were repeated three times and the results averaged. Controls included wells containing bacteria+PBS, HRBC+PBS, bacteria+test compound, HRBC+test compound, and bacteria+HRBC.

### Results

The final concentration at which anti-adhesion activity could be detected in SP4 was 0.23 mg/mL. The value of 0.23 mg/mL is consistent with results obtained from other juice-based column-extracted cranberry powders we have tested. A direct comparison with organic cranberry juice powder (30 mg/mL), high PAC level cranberry extracts (0.47 mg/mL), low PAC level cranberry extracts (3.5-7.5 mg/mL), bacterial anti-adhesion activity of the whole Urell®/Ellura™ cranberry powder (measured by MRHA activity, 0.47 mg/mL) indicates that SP4, with 0.23 mg/mL possesses a high antiadhesion activity towards uropathogenic P-fimbriated Escherichia coli.

### Example 9. Pre-Clinical Double-Blind Controlled Study on the Treatment of Urinary Tract Infection with the Formulation SP4

To assess the effect of the formulation SP4 of Example 3, we recruited participants from a population of volunteers (10 women) involved in studies performed by the Farmacia Antoniana (San Gillio, Italy) under the supervision of medical doctors. Informed consent was obtained. The inclusion criteria included any woman at least 18 years of age to over 51 years of age with at least 2 culture-documented symptomatic UTIs in the calendar year prior to recruitment. The choice of volunteers was completely balanced, and volunteers with known anatomical abnormalities (posterior urethral valves, neurogenic bladder, or any urinary obstruction) were excluded from this study. Urinary infection was defined as a positive culture of a midstream sample with a uropathogenic bacterium at 10′ colony forming unit (CFU)/mL in symptomatic volunteers with no more than two species of organisms present. We accepted lower counts (10⁴ CFU/mL) if the volunteer had typical symptoms of UTI and positive white blood cells and/or nitrites on urine analyses. Specific symptoms and signs included pain before, during, or after micturition; increased frequency of micturition; pain in abdomen; hematuria; foul smell; and signs of common sickness (fever>37.9° C. or 1.5° C. above baseline, temperature, chills, nausea, and vomiting). Nonspecific symptoms were considered anorexia, fatigue and reduced mobility, and signs of delirium (e.g., confusion and deterioration in mental or functional status). Asymptomatic bacteriuria was not considered an end point. After explaining the study and obtaining consent, patients were assigned to the placebo group or experimental randomized groups. The randomization was concealed.

Administration was achieved by using capsules containing 600 mg of the formulation SP4 of Example 3 (equivalent to 329 mg total PACs and 168 mg PAC-A mg PAC-A). Placebo capsules were indistinguishable in color, taste, and appearance, consisting of all elements above without formulation of Example 3 and colored with azorubine. The experimental group (7 females) received 1 capsule containing 168 mg PAC-A twice a day (morning and evening) for 7 days, and the placebo group (3 females) was given the same number of capsules with no PACs. A score (from 0, representing no effect, to 10, representing a maximum effect of formulation of Example 3 in preventing UTI) was recorded for all volunteers. The dose was calculated based on previous clinical trials. The administration was performed for 7 days; during this time, the volunteers were followed with alternating visits and telephone calls every 2 days. At the end of the treatment period, a urine sample was sent for urine analysis. To avoid contamination, the volunteers were asked to not use antibiotics or any other product for the duration of the study, with the exception of the placebo group, in which volunteers were asked to immediately report on symptoms. In the latter case, they were asked to use the antibiotic prescribed by the medical doctor and to interrupt the placebo administration. The attending urologists, outcome assessor and statistician were all blinded to the group allocations.

In all volunteers, the infection prior to recruitment was due to E. coli in 90%, other enteric Gram-negative bacilli in 8%, and more than 1 type of bacteria in 2% of the volunteers. The following table shows the volunteers' demographic and baseline characteristics.

TABLE 18
Volunteers baseline characteristics.

| | Experimental Group, Placebo Group, | |
|---|---|---|
| | Formulation SP4 of n = 3 | |
| | Example 3 | |
| Variables | Administration, n = 6 | |
| Demographics | | |
| Age range | | |
| 60-90 | 6 | 3 |
| Baseline characteristics | | |
| Acute UTI, (%) | 6 (100) | 3 (100) |
| Bladder and bowel | 6 (100) | 3 (100) |
| dysfunction, (%) | | |
| Average UTIs in years | 2.2 | 2.1 |
| prior to treatment | | |
| Number of capsules (days) | 2 (7) | 2 (7) |

Abbreviations: UTI, urinary tract infections.

No dropout occurred in treatment, whereas all 3 placebo dropped out due to acute pain. The median follow-up time in both groups was 4 weeks.

The mean capsule intake was 98% (95% CI: 96.6-98.6%) and was similar between the experimental and placebo groups. After 7 days of formulation SP4 of Example 3 most of the placebo group were unable to recover from UTI. Eventually, all placebo volunteers had to be treated with antibiotics (Monuril®, trometamol salt of fosfomycin) to reduce pain. A significant difference was found between the placebo and subjects taking formulation SP4 of Example 3. Finally, considering the CFU/mL counts from the urocultures, a significant difference (P<0.001) was found in the comparison between the experimental group and the placebo group (P<0.001, Bonferroni Adjusted P<0.001).

Overall, these results show that the administration of formulation SP4 of Example 3 significantly ameliorated UTI in the treatment group.

The following are examples of urine analyses performed on some of the volunteers:
CASE 1 volunteer born Apr. 14, 1962; the total bacterial load on Nov. 11, 2021 was 10,000,000.00 CFU/ml. On Nov. 25, 2021 the urine analyses show negative bacterial counts.
CASE 2 volunteer born Mar. 23, 1934; the total bacterial load on Aug. 23, 2021 was 100,000.00 CFU/ml. On Sep. 23, 2021 the urine analyses show negative bacterial counts.
CASE 3 volunteer born May 5, 1945; the total bacterial load on Sep. 17, 2021 was 1,000,000.00 CFU/ml. On Oct. 10, 2021 the urine analyses show negative bacterial counts.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A formulation comprising PAC-enriched compositions extracted from each of Vitis vinifera, Arachis hypogaea, Pinus pinaster, Vaccinium vitis-idaea.

2. The formulation of claim 1, wherein the formulation comprises
1-25 wt % Vitis vinifera PAC-enriched composition;
40-85 wt % Arachis hypogaea PAC-enriched composition;
1-35 wt % Pinus pinaster PAC-enriched composition; and
5-45 wt % Vaccinium vitis-idaea PAC-enriched composition.

3. The formulation of claim 1, wherein the formulation contains 500-600 mg/g of PACs.

4. The formulation of claim 3, wherein the formulation has a peanut allergen content of 1.5 μg or less.

5. The formulation of claim 1, wherein the PAC-enriched compositions comprise dimeric, trimeric, tetrameric, and pentameric PACs.

6. The formulation of claim 1, wherein the PAC-enriched compositions comprises a higher percentage of A-type PACs as compared to a percentage of A-type PACs in the plant material prior to extraction.

7. The formulation of claim 1, wherein each of the PAC-enriched compositions were extracted using 90-100 vol % ethanol or 90-100 vol % acetone.

8. The formulation of claim 1, wherein the formulation is in a solid dosage form selected from a tablet, capsule, or softgel.

9. A method for preparing a proanthocyanidin (PAC)-enriched composition, comprising
mixing a plant material containing PACs with an extraction solvent comprising 90-100 vol % ethanol or 90-100 vol % acetone to provide a mixture;
filtering the mixture to provide a filtrate; and
evaporating the filtrate to provide a dried PAC-enriched composition.

10. The method of claim 9, further comprising crushing the dried PAC-enriched composition to provide a powder.

11. The method of claim 9, wherein the plant material is obtained from one or more species selected from the group consisting of Vaccinium macrocarpon, Vitis vinifera, Arachis hypogaea, Pinus pinaster, Vaccinium vitis-idaea.

12. The method of claim 9, wherein the extraction solvent further comprises an acid.

13. The method of claim 9, wherein a ratio of extraction solvent to plant material is between 3:1 and 8:1.

14. The method of claim 9, wherein the mixing step is performed for 10-60 minutes.

15. The method of claim 9, wherein the mixing step is performed at a temperature of 10-30° C.

16. The method of claim 9, wherein the evaporating step is performed using vacuum evaporation.

17. The method of claim 9, wherein the evaporating step is performed using vacuum evaporation to provide a dense liquid followed by drying the dense liquid at a temperature of 40-90° C.

18. The method of claim 9, wherein the evaporating step is performed via spray drying.

19. The method of claim 9, wherein the PAC-enriched composition comprises dimeric, trimeric, tetrameric, and pentameric PACs.

20. The method of claim 9, wherein the PAC-enriched composition comprises a higher percentage of A-type PACs as compared to a percentage of A-type PACs in the plant material prior to extraction.

21. The method of claim 9, further comprising determining a PAC content in the plant material before the mixing step.

22. A PAC-enriched composition prepared by the method of claim 9.

23. The composition of claim 22, wherein the composition is in a solid dosage form selected from a tablet, capsule, or softgel.

24. A method of inhibiting replication of viral particles, comprising contacting the viral particles with the formulation of claim 1.

25. The method of claim 23, wherein the viral particles are a virus selected from the group consisting of influenza virus, respiratory syncytial virus, coronavirus, ebola virus, and human immunodeficiency virus.

26. A method of treating a viral infection in a subject in need thereof, comprising administering to the subject a therapeutically effective amount of the formulation of claim 1.

27. The method of claim 26, wherein the viral infection is caused by a virus selected from the group consisting of influenza virus, respiratory syncytial virus, coronavirus, ebola virus, and human immunodeficiency virus.

28. A method of inhibiting adhesion of bacteria to cells, comprising contacting the bacteria with the formulation of claim 1.

29. The method of claim 28, wherein the bacteria is selected from one or more of Escherichia coli, Coagulase negative Staphylococci, Enterococcus Spp., Helicobacter pilorii, Candida albicans, and Non-albicans Candida Spp.

30. A method of treating a bacterial infection in a subject in need thereof, comprising administering to the subject a therapeutically effective amount of the formulation of claim 1.

31. The method of claim 30, wherein the bacterial infection is a urinary tract infection caused by one or more of Escherichia coli, Coagulase negative Staphylococci, Enterococcus Spp., Helicobacter pilorii, Candida albicans, and Non-albicans Candida Spp.
